(19) United States
(12) Patent Application Publication
(10) Pub. No.: US 2015/0238697 A1
(43) Pub. Date: Aug. 27, 2015

(54) Syringes Having Tactile Labeling Elements

(71) Applicant: Andersen-Michaud, LLC, Presque Isle, ME (US)

(72) Inventors: Kathleen Dahlgren Michaud, Perham, ME (US); Michelle Andersen, Presque Isle, ME (US)

(21) Appl. No.: 14/509,909

(22) Filed: Oct. 8, 2014

Related U.S. Application Data
(60) Provisional application No. 61/931,573, filed on Jan. 25, 2014.

(51) Int. Cl. A61M 5/31

(52) U.S. Cl. CPC A61M 5/31; A61M 2205/6045

(57) ABSTRACT

Syringes having various tactile and visual differentiation features are provided. Elements of differentiation ribs of various shapes and configurations are located on the syringe tube. The different configurations provide tactile cues and to some extent visual cues that enable a user to quickly and easily identify different syringes and accordingly the syringe contents thereby encouraging greater precision in the delivery of correct medications.

### BACKGROUND

The health care industry continues to search for ways to reduce medical errors. Medical errors cause tens of thousands of patient injuries and deaths every year. One class of these errors is medication delivery error that is, giving a patient a wrong medication or a wrong dosage of a medicine.

One subset of medication delivery error involves injected medications. Typically medication is injected via a syringe. A typical syringe is a simple pump consisting of a plunger that fits tightly inside a cylindrical tube, also referred to as a “barrel”. The plunger can be pulled and pushed inside the cylindrical tube. The pulling and pushing allow the syringe to take in and expel a liquid medication through an orifice at an open end of the tube. For medical uses, the open end of the syringe is typically fitted with a needle. Another typical medical use for the syringe is to inject medication into an intravenous fluid infusion set. Generally, syringes are prepared, that is, filled with a medication close to the time that the medication is to be delivered. There is, however, typically a time gap, or delay, and sometimes a personnel gap between the time that the syringe is prepared and the time when the medication is injected into a patient. In these gaps, syringes can become confused by the person who performs the injection and the wrong medication can be delivered.

One attempt to prevent medical error in injected medications is syringe labeling. Types of syringe labeling include both affixing labels to the syringe and writing directly on the syringe, either onto a “labeling strip” already applied to the syringe or directly onto the syringe barrel with a pen. Compliance with syringe labeling has been generally found to be low apparently because of the time and effort involved in the labeling process.

It remains desirable to have innovative medication delivery mechanisms that can reduce the propensity for medication delivery error, that can be readily adopted by health care institutions, and that have convenience and ease of use that will encourage compliance.

### SUMMARY

The present invention is directed to a syringe adapted to inhibit medication delivery error thereby addressing the problems articulated above.

Syringes having various tactile and visual differentiation features are provided. The tactile and visual differentiation elements act as a labeling system and an alert to the user. For example, the user may be alerted to a “high-alert” medication by the tactile surface elements on the syringe barrel. The visual and tactile differentiation can be used to alert the user to different medications in a group of filled syringes. Essentially, the tactile and visual differentiation is a way of alerting the user to pay attention and is a quick cue to syringe contents.

The tactile and visual differentiation elements include ribbing on the tube, or “barrel” of the syringe. Alternatively, the tube has tactile strips. In a system employing the embodiments presented here, the user is alerted to the type of medication in the syringe .

In one embodiment, the vertical ribs on the outside of the tube create the impression of a square or triangular shape on the hand holding the syringe. In further alternative arrangements, the syringe tube is hexagonal or decahedronal or dodecahedronal in cross-section.

In an alternative embodiment, ribs arranged on the outside surface of the syringe tube provide a tactile difference between syringes. In a first arrangement, the outside surface of the tube has at least one vertical rib. In a second arrangement, the outside surface of the tube has at least one horizontal rib, also referred to as a “circumferential rib”.

The ribs provide both visual as well as tactile differentiation to syringes. This differentiation provides a safety improvement in that easily distinguishable differences between syringes will discourage medicine delivery errors. Specifically, medications designated as “high-alert” medications such as muscle relaxants, narcotics, and insulin could be more easily distinguished from other medications. A differently configured syringe would be a tactile indicator to a health care provider, often working in an environment that is hurried and full of distractions, of the type of drug that the syringe holds. Further, syringe differentiation would be a strong safety enhancement in the production and use of pre-filled syringes which are expected to be used for a wide range of medications, including vaccines, anticoagulants, anti-infectives, anti-inflammatory agents, hematological agents, multiple sclerosis therapies, hormone therapies, obstetric agents, cancer therapies, pain relievers, vasopressors, local anesthetics, and hypnotic agents.

In another alternative embodiment, the ribs on the syringe tube are provided as part of the syringe labeling. Together these innovations have the potential to add an extra margin of safety for patients against the potentially harmful consequences of being administered a wrong drug. Embodiments of the present invention provide the advantages of reducing medication delivery errors and their associated costs, both human and financial.

Another embodiment is a barrel for a syringe where the barrel is a tube that has an outer surface. This embodiment further includes a tactile element on the outer surface configured such that the tactile element acts as a tactile label for the syringe whereby a user of the syringe is able to perceive an alert to the contents of the syringe. A barrel with a tactile labeling element on a syringe could be used to alert a syringe user to a particular medication contained in the syringe thereby tending to reduce medical error.

In a first alternative embodiment, the tactile element is a plurality of vertical ribs. In a one arrangement, the plurality of ribs are arranged and configured to create an impression of a particular geometric figure in cross-section such as a square or a triangle. This embodiment retains the underlying configuration of a syringe with a cylindrical barrel and further including a non-rounded shape superimposed over the familiar arrangement. This embodiment provides the user with a tactile alert to the user differentiating the syringe of the present embodiment from a conventional syringe.

Another embodiment is a barrel for a syringe where the barrel is a tube that has an outer surface. This embodiment further includes a tactile element of circumferential ribs on the outer surface configured such that the tactile element acts as a tactile label for the syringe whereby a user of the syringe is able to perceive an alert to the contents of the syringe. In an alternative arrangement, the plurality of circumferential ribs are located and arranged as a plurality of groups on the outer surface of the syringe barrel.

Another embodiment is a barrel for syringe where the barrel is a tube that has an outer surface. The outer surface of the tube includes a tactile element where the tactile element is a spiral ridge.

Another embodiment is a barrel for a syringe where the barrel is a tube that has an outer surface. The outer surface includes a tactile element that is a plurality of bumps. In an alternative arrangement, the tactile element is a plurality of dimples.

Another embodiment is a syringe for delivery of a medication where the syringe includes a barrel having a cylindrical tube. The cylindrical tube has an outer surface. The outer surface includes a tactile element configured such that the tactile element acts as a tactile label for the syringe whereby a user of the syringe is able to perceive an alert to the contents of the syringe.

In one alternative embodiment of the syringe, the tactile element is a plurality of vertical ribs. In one arrangement, the plurality of vertical ribs are arranged and configured to create an impression of a particular geometric figure in cross-section such as a square or a triangle.

Another embodiment is a syringe for delivery of a medication where the syringe includes a barrel having a cylindrical tube. The cylindrical tube has an outer surface. The outer surface includes a tactile element where the tactile element is a plurality of circumferential ribs.

Another embodiment is a syringe for delivery of a medication where the syringe includes a barrel having a cylindrical tube. The cylindrical tube has an outer surface. The outer surface includes a tactile element where the tactile element is a spiral ridge.

Another embodiment is a syringe for delivery of a medication where the syringe includes a barrel having a cylindrical tube. The cylindrical tube has an outer surface. The outer surface includes a tactile element where the tactile element is plurality of bumps. In an alternative embodiment, the tactile element is a plurality of dimples.

In another embodiment, the syringe includes a plunger having a thumb plate. The thumb plate further includes a second tactile element configured such that the second tactile element forms a second non-textual tactile label. From this second non-textual tactile label, a user of the syringe is able to perceive an alert to contents of the syringe. In one arrangement, the second tactile element is a geometric configuration of the thumb plate. In a second arrangement, the second tactile element is at least one raised structure locating on the upper surface of the thumb plate.

In another embodiment, the syringe includes an adapter cap. The adapter cap includes a third tactile element configured such that it forms a third non-textual tactile label. Through this third non-textual label, a user of the syringe is able to perceive an alert to contents of the syringe.

In another embodiment, the syringe includes a barrel, a plunger with a thumb plate and an adapter cap. The barrel includes a tactile element that forms a non-textual tactile label. The thumb plate further a second tactile element formed and configured as a second non-textual tactile label. The adapter cap further include a third tactile element formed and configured as a third non-textual tactile label. The tactile elements on the barrel, the thumb plate and adapter cap match providing the user with an alert to contents of the syringe.

Another embodiment is a syringe for delivery of a medication where the syringe includes a barrel having a cylindrical tube. The cylindrical tube has an outer surface. The outer surface includes a tactile element where the tactile element is a flexible sheet with tactile labeling elements. The flexible sheet is affixed to the outer surface of the tube.

The present invention together with the above and other advantages may best be understood from the following detailed description of the embodiments of the invention illustrated in the drawings, wherein:

### DRAWINGS

FIG. 1 is a side view of a syringe with a tube having a plurality of vertical ribs according to principles of the invention;

FIG. 2 is a cross-sectional view of the syringe of FIG. 1;

FIG. 3A is a cross-sectional view of a first alternative embodiment of ribs on a syringe tube according to principles of the invention;

FIG. 3B is a cross-section view of a second alternative embodiment of ribs on a syringe tube according to principles of the invention;

FIG. 4 is a side view of a syringe with a tube having an alternative type of vertical rib on the syringe tube according to principles of the invention;

FIG. 5 is a side view of a syringe having circumferential ribbing along the syringe tube according to principles of the invention;

FIG. 6 is a side view of a syringe having a spiral ridge according to principles of the invention;

FIG. 7 is a side view of a syringe having tactile strips according to principles of the invention;

FIG. 8 is an illustration of a label having ribs according to principles of the invention,

FIG. 9 is a cross-section view of a syringe where the ribs are arranged to form a square shape around the syringe barrel according to principles of the invention;

FIG. 10 is a cross-section view of a syringe where the ribs are arranged to form a triangle shape around the syringe barrel according to principles of the invention;

FIG. 11 is a side view of a syringe having a surface with a plurality of protrusions according to principles of the invention;

FIG. 12 is a top view of an embodiment of a thumb plate according to principles of the invention;

FIG. 13 is a top view of an alternative embodiment of a thumb plate according to principles of the invention;

FIG. 14 is a top view of another alternative embodiment of a thumb plate according to principles of the invention;

FIG. 15 is a top view of another alternative embodiment of a thumb plate according to principles of the invention;

FIG. 16A is a top view another alternative embodiment of a thumb plate according to principles of the invention;

FIG. 16B is a side view of the thumb plate of FIG. 16A;

FIG. 17 is a top view of another alternative embodiment of a thumb plate according to principles of the invention;

FIG. 18 is a top view of another alternative embodiment of a thumb plate according to principles of the invention;

FIG. 19 is a top view of another alternative embodiment of a thumb plate according to principles of the invention;

FIG. 20 is a top view of another alternative embodiment of a thumb plate according to principles of the invention;

FIG. 21 is a top view of another alternative embodiment of a thumb plate according to principles of the invention;

FIG. 22 is a top view of another alternative embodiment of a thumb plate according to principles of the invention;

FIG. 23 is a top view of another alternative embodiment of a thumb plate according to principles of the invention;

FIG. 24 is a top view of another alternative embodiment of a thumb plate according to principles of the invention;

FIG. 25 is a top view of another alternative embodiment of the thumb plate according to principles of the invention;

FIG. 26 is a top view of another alternative embodiment of the thumb plate according to principles of the invention;

FIG. 27 is a top view another alternative embodiment of a thumb plate according to principles of the invention; and

FIG. 28 is a side view of an embodiment of a syringe including an adapter cap according to principles of the invention.

### DESCRIPTION

Embodiments of the present invention include syringes adapted to reduce instances of medication delivery error. Syringes according to embodiments of the present invention have barrels that are variously configured such that they provide non-textual tactile differentiation. . The tactile elements on the syringes are formed and configured such that the tactile elements form a non-textual tactile label. The tactile elements provide alerts to users of the syringes to be aware of the syringe contents. The different syringe configurations also provide visual differentiation beyond text labeling. The syringes are typically in the range of sizes from 1 milliliter to 60 milliliters, however the present invention is not limited to this range.

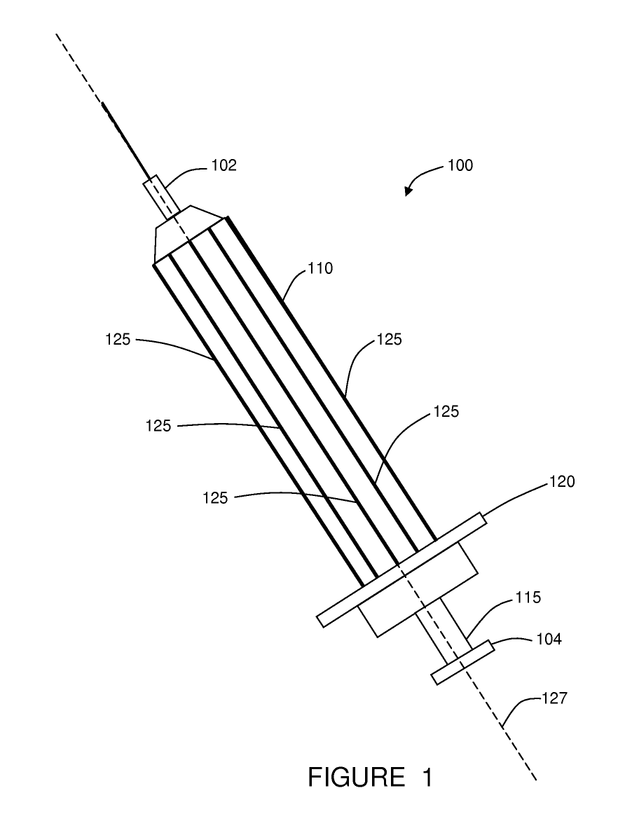

FIG. 1 is a side view of a syringe according to one embodiment. The syringe 100 has a cylindrical tube 110. The tube 110 is also referred to as a “barrel.” The tube 110 includes a flange 120 at one end and an adapter 102 at the other end. The adapter 102 in some arrangements holds a needle as shown in FIG. 1. In alternative arrangements, the adapter 102 is mateable with an intravenous fluid infusion set. The syringe 100 further has a plunger 115. The plunger 115 is inserted into the flanged end of barrel 110 and is configured to slide inside the barrel 110. The end of the plunger 115 inside has a plunger seal (not shown in this figure) to maintain a tight fit between the plunger and the inner surface of the barrel. The end of the plunger 115 outside the barrel has a thumb plate 104.

Typically, a syringe user grips the barrel between the second and third fingers of one hand and presses on the plunger with the thumb of that hand to push a fluid out of the adapter end of the syringe.

The syringe 100 has a plurality of vertical ribs 125, also referred to as “ridges”, arranged on the outer surface of the tube 110 around the tube's circumference. The ribs 125 are substantially parallel to the vertical axis 127 of the tube 110. Further, the ribs 125 are arranged substantially equidistantly around the tube 110.

In a first arrangement, the ribs 125 are formed as part of the tube 110 in, for example, an extrusion process. In a second arrangement, the ribs 125 are applied to the tube 110. The ribs are applied, for example, as part of a flexible sheet affixed to the outer surface of the tube 110. In the flexible sheet, the ribs form a part of a medication label to be located on the outer surface of the tube thereby providing both a tactile and visual differentiation to the syringe. The ribs may also be applied individually. The present embodiment is not limited to those methods of forming the ridges provided here.

The ribs 125 provide a tactile alert to the person using the syringe to deliver medication. The ribs 125 are a surface element on the syringe barrel that differentiates the barrel of the present embodiment from a conventional syringe having no tactile differentiation elements. The user of the syringe of the present embodiment would have a tactile alert when handling the syringe beyond any text labeling. If, for example, “high-alert” medications are used only in syringes having a tactile alert on the barrel and other types of medications are used in conventional syringes, the tactile alert would tend to make a user aware of the high-alert medication and medication delivery errors would tend to be averted. The tactile differentiation is not related to any text, for example, in the way that Braille represents letters and can be used to form words. The tactile differentiation provides a quick and simple alert to the user. In addition to the non-textual tactile alert, the ribs 125 provide a visual difference between the syringe of the present embodiment and conventional syringes. The visual difference may be enhanced by ribs having a color different from that of the syringe barrel.

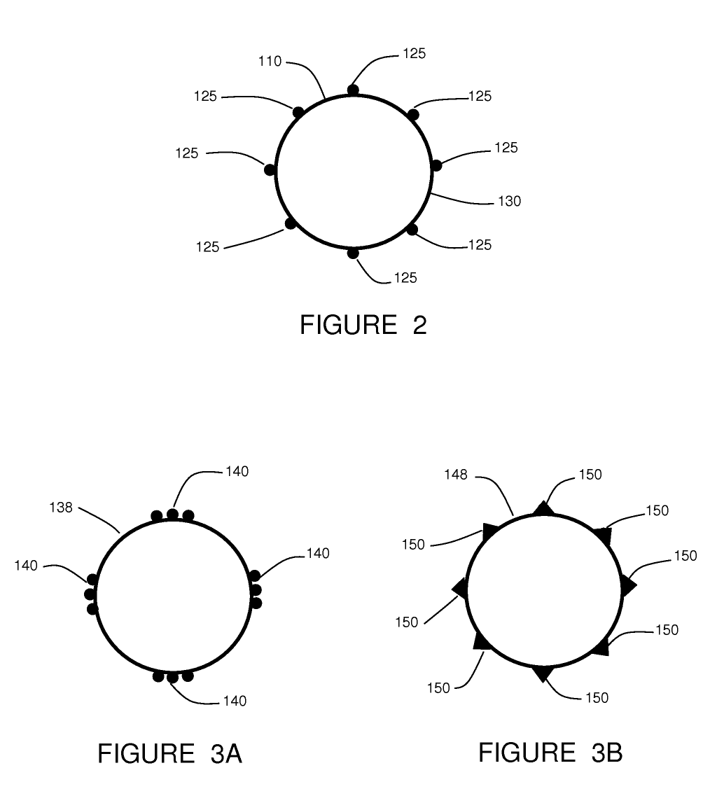

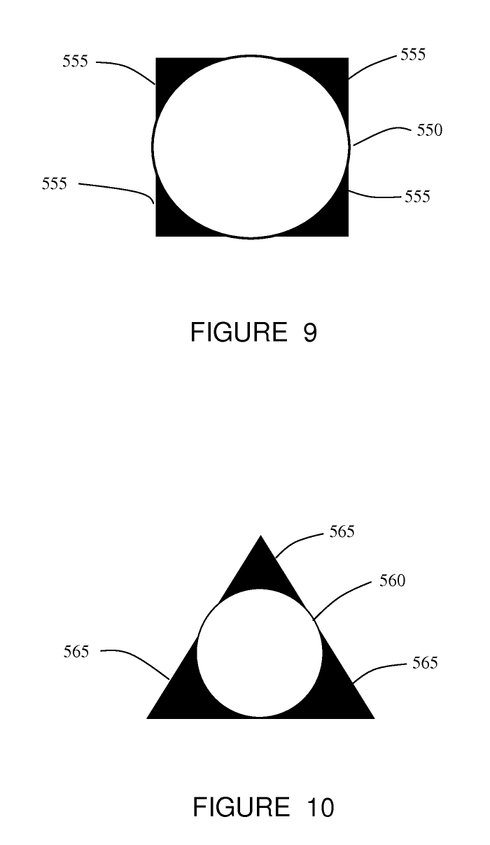

FIG. 2 is a cross-sectional view of the syringe of FIG. 1. The tube 110 of the syringe has ribs 125 arranged around the outer surface 130. In this embodiment, the ribs 125 are rounded ridges. Alternative configurations and forms are possible within the scope of the invention. For example, in one alternative embodiment, there is only one vertical rib. In another arrangement, the vertical ribs are arranged in groups, for example, the groups of three ribs shown in FIG. 3A. In other alternative embodiments, for example, the vertical ribs on the outside of the tube create the impression of a square or triangular shape in the hand holding the syringe. These embodiments are shown in FIGS. 9 and 10 respectively and will be described in greater detail below. In further alternative arrangements, the syringe tube has additional vertical ribs on the outside surface such that it creates the impression of a hexagonal or decahedronal or dodecahedronal shape in the hand holding the syringe. Examples of further alternative embodiments are shown in FIGS. 3A and 3B.

FIG. 3A is a cross-sectional view of an alternative embodiment of ribs on a syringe tube 138. In this embodiment, groups 140 of vertical ribs are arranged around the outer surface of the syringe tube 138. This alternative arrangement of ribs provides both tactile and visual differentiation both from syringes without ribbing and from the embodiment shown in FIGS. 1 and 2.

FIG. 3B is a cross-sectional view of a further alternative embodiment of a syringe having ribs 150 on the outer surface of the syringe tube 148. In this embodiment, the ribs 150 are not rounded and instead are triangular in cross-section and arranged in a manner where an edge is presented to a user gripping the syringe.

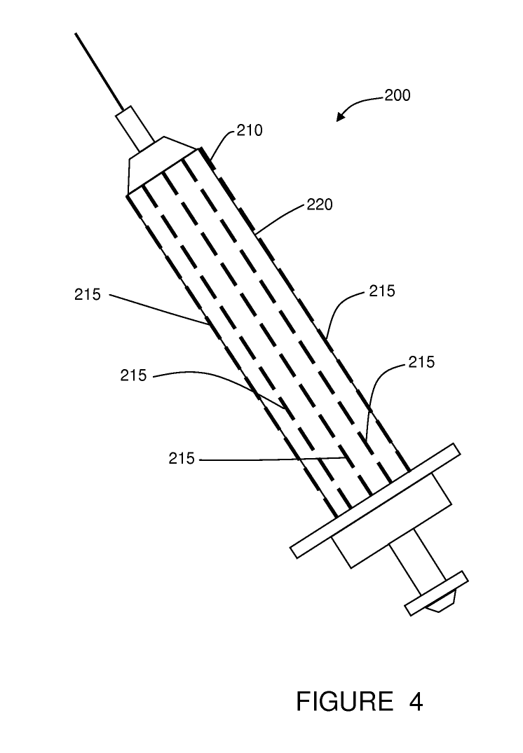

FIG. 4 is a side view of a syringe 200 with a tube 210 having an alternative type of vertical rib 215 on the syringe surface 220. The vertical ribs 215 have gaps along each rib. This arrangement provides tactile differentiation as well as visual differentiation.

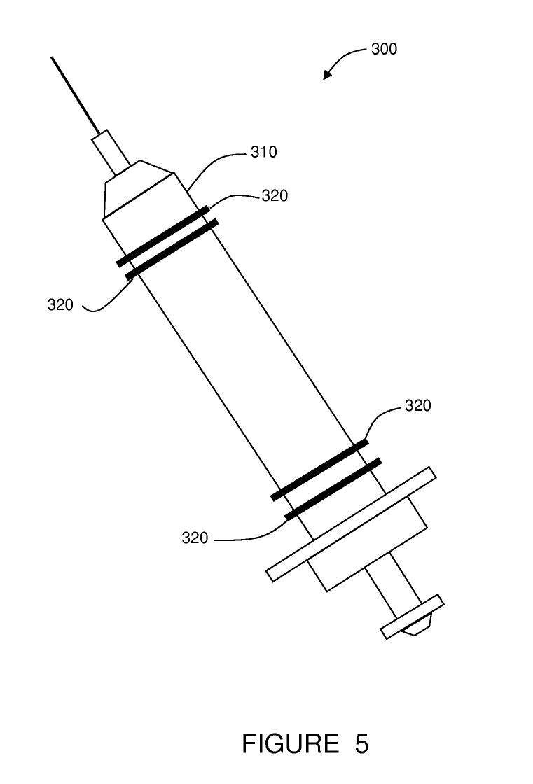

FIG. 5 is a side view of a syringe 300 having an alternative rib arrangement. In this embodiment, circumferential ribs 320 are located on the outer surface of the syringe tube 310. In this embodiment, only four ribs are shown. The ribs 320 are arranged in two groups. A first group is located closer to the needle end of the tube 310. A second group is located closer to the plunger end of the tube 310. Alternative embodiments of rib number and location are possible. The alternative embodiments provide both tactile and visual differentiation from other configurations.

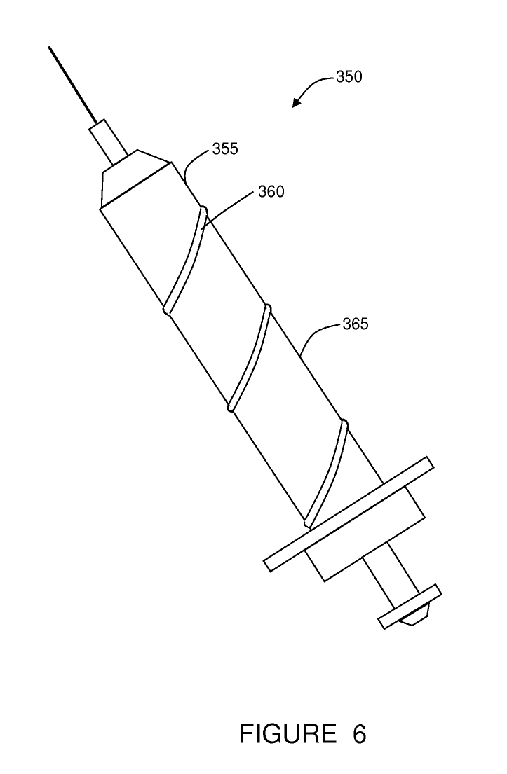

FIG. 6 is a side view of an alternative embodiment of a syringe 350. In this embodiment, the syringe tube 355 has a spiral ridge 360 on the surface 365 of the tube 355.

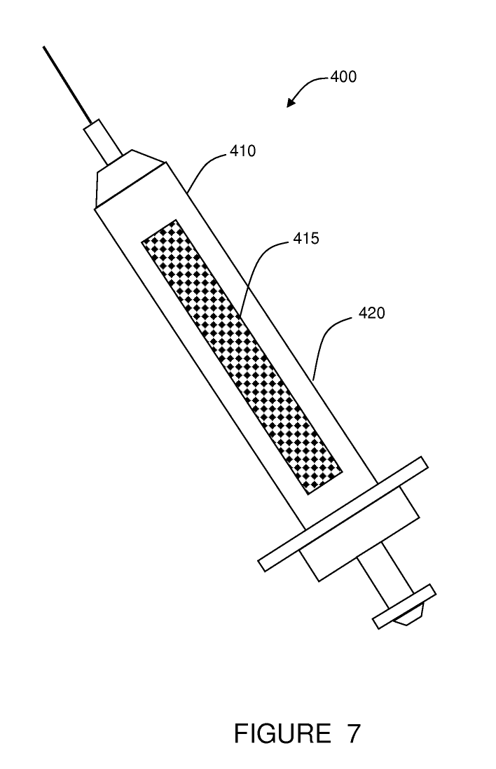

FIG. 7 is a side view of an alternative embodiment of a syringe 400. In this embodiment, the syringe tube 410 has a tactile strip 415 located on the surface 420 of the tube 410. The tactile strip 415 has, for example, a roughened surface that is semi-random like sandpaper. Alternatively, the tactile strip 415 has a regularly roughened surface having a pattern such as a diamond pattern. Other types of roughening are possible within the scope of the invention. In alternative embodiments, the syringe tube 410 has more than one tactile strip 415. The tactile strip may be formed as a part of the manufacture of the tube. Alternatively, the tactile strip may be applied after the tube is formed, for example as a separate sheet, or for example, through a printing process. In a further alternative embodiment, the tactile strip incorporates medication information and acts as a text label in addition to a tactile label.

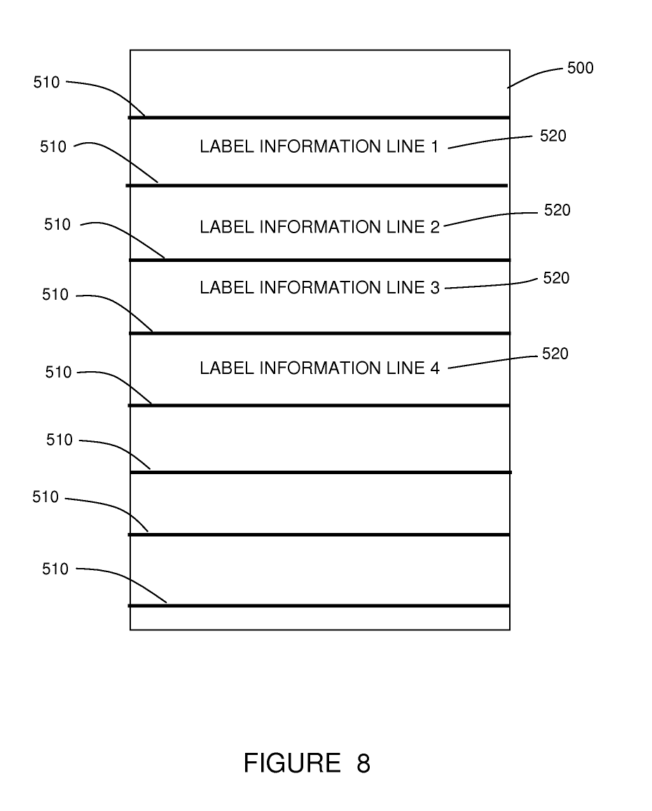

FIG. 8 is an illustration of a sheet having ribs according to one embodiment. The sheet 500 is made of a flexible material such as a plastic. The sheet 500 includes a number of ribs, or “ridges”, 510. The sheet 500 further includes text information 520 so that it may also function as a text label. The sheet is also capable of being adhered to plastic or glass so that it is capable of being adhered to a syringe tube to provide both visual and tactile cues about the contents of the syringe. The sheet 500 shown in this embodiment has the ribs equivalent to those shown in FIG. 1, however, other types of surface element can be accomplished on the sheet as well such as the circumferential rib, the spiral rib, and the tactile strip. The differentiating tactile surface elements available on the sheet 500 are not limited to those listed above. Other types of surface element are within the scope of the invention.

FIG. 9 is a cross-sectional view of an alternative embodiment of ribs on a syringe tube 550. In this embodiment, four vertical ribs 555 are arranged around the outer surface of the syringe tube 550. The four vertical ribs 555 are arranged and configured around the syringe tube 550 such that the impression on the hand of the syringe user is that of a syringe that is square in cross-section.

FIG. 10 is a cross-sectional view of an alternative embodiment of ribs on a syringe tube 560. In this embodiment, three vertical ribs 565 are arranged and configured around the syringe tube 560 such that the impression on the hand of the syringe user is that of a syringe that is triangular in cross-section.

The configurations shown in FIGS. 9 and 10 are merely exemplary. Other rib configurations forming other shapes in cross-section such as a hexagon or an octagon are possible within the scope of the invention.

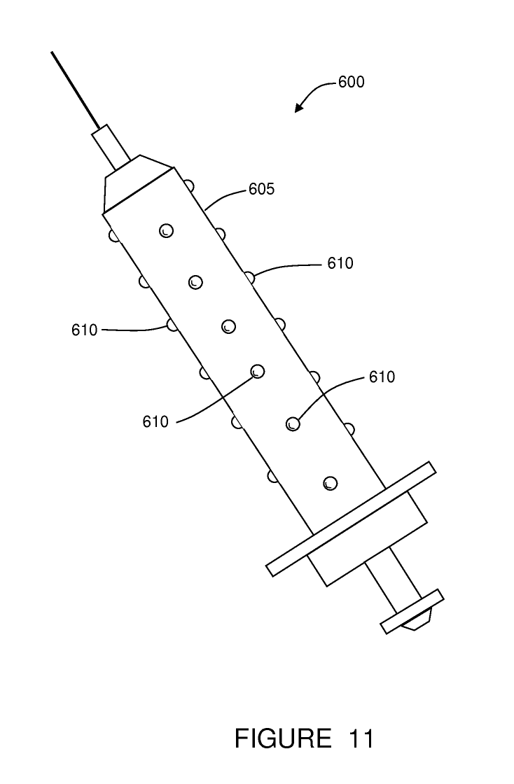

FIG. 11 is a side view of an alternative embodiment of a syringe 600. In this embodiment, the syringe 600 has a surface 605 including a plurality of protrusions, also referred to as “bumps” 610. The bumps 610 in this embodiment are formed on the surface 605 of the syringe barrel. In alternative embodiments, the bumps 610 are part of a label applied to the tube for example with adhesive. In an alternative arrangement, the bumps are concave, or “dimples.” Various arrangements of dimples or bumps are possible in order to provide tactile differentiation. The placement, distance between, number and arrangement of bumps are all possible variations on the syringe tube surface. For example, there may be in one arrangement a single vertical line of bumps on the surface of the syringe. In an alternative arrangement, there are two lines of bumps. Further alternative shapes of bumps are within the scope of the invention. For example, the bumps may be round, oval, square, triangular, pointed or pyramid-shaped. The alternatives provided here are merely exemplary. The form and configuration of the bumps is not limited to those described here. The bumps may also be of a different color than the syringe tube thereby providing a visual differentiation element as well as a tactile differentiation element.

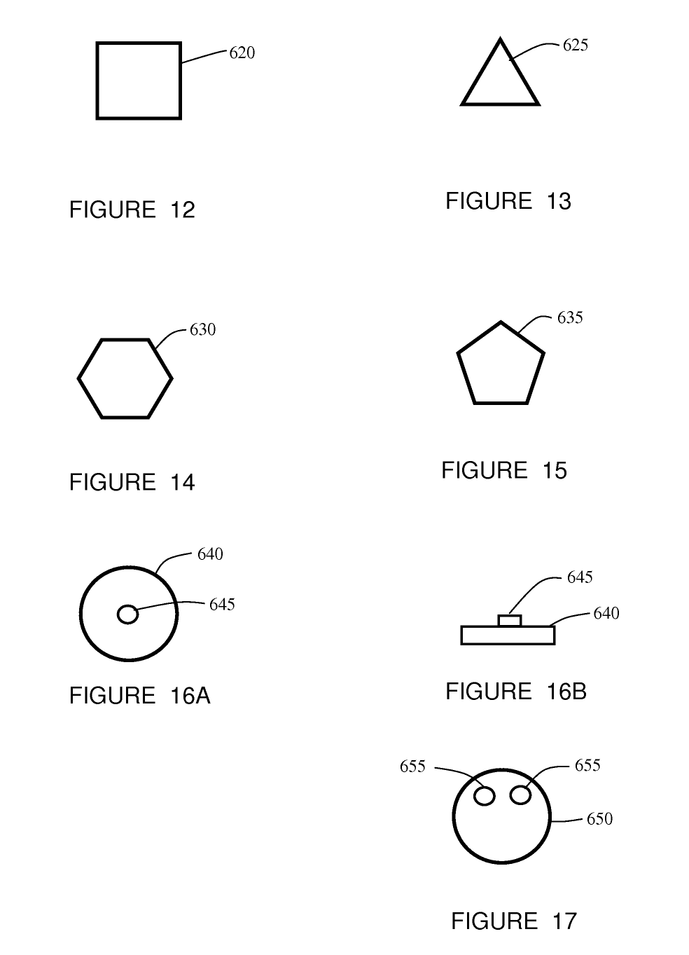

The thumb plate of a syringe is another location where tactile differentiation may be effectively used to provide non-textual cues to syringe users about the syringe's contents. FIGS. 12-27 show embodiments of various embodiments of thumb plates with tactile differentiation. The tactile differentiation of the thumb plate could be used alone or in combination with differentiation on the surface of the syringe barrel.

FIG. 12 is a top view of a thumb plate 620 that is substantially square in shape. FIG. 13 is a top view of a thumb plate 625 that is substantially triangular in shape. FIG. 14 is a top view of a thumb plate 630 that is substantially a six-sided polygon in shape. FIG. 15 is a top view of a thumb plate 635 that is substantially hexagonal in shape. The thumb plate shapes provided here are merely exemplary. Other shapes are possible within the scope of the invention.

FIG. 16a is a top view of a thumb plate 640 that is substantially circular. FIG. 16b is a side view of the thumb plate 645 shown in FIG. 16a. The thumb plate 640 has a top surface. A round protrusion 645 is located on the top surface. In this embodiment, the protrusion has a radius that is somewhat smaller than that of the thumb plate. In FIG. 21, a round protrusion 695 is located on the top surface of the thumb plate 690. The protrusion in FIG. 21 has a radius that is not much smaller than the radius of the thumb plate 690.

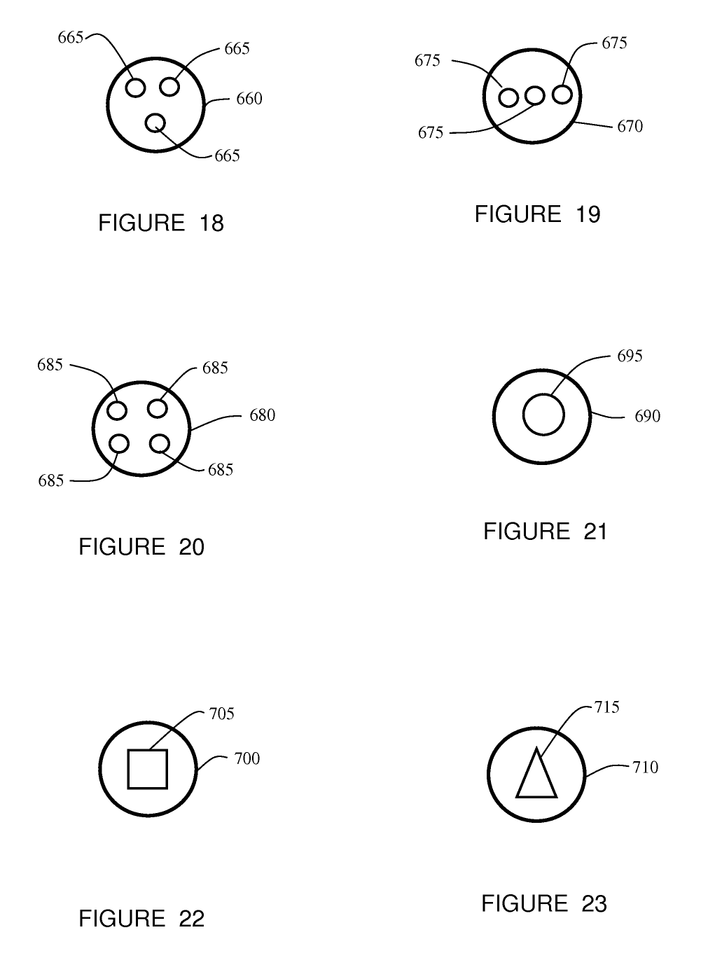

FIG. 17 is a top view of a thumb plate 650 having two protrusions 655 on its top surface. The two protrusions are located off the center of the thumb plate 650 in this embodiment however, the protrusions may be centered in an alternative arrangement. FIG. 18 and FIG. 19 are top views of thumb plates 660, 670. Each thumb plate 660, 670 has three protrusions 665, 675. The protrusions 665 in FIG. 18 are centered and are arranged in a triangular shape. The protrusions 675 are centered but are arranged in a line. FIG. 20 is a top view of a thumb plate 680 having four protrusions 685. The protrusions 685 are centered and arranged in a square shape. The arrangements discussed here are merely exemplary. Other arrangements of the protrusions are possible within the scope of the invention.

FIGS. 21, 22 and 23 are top views of thumb plates 690, 700, 710 with a single relatively large protrusion on the top surface of each. FIG. 21 has a circular protrusion 695. FIG. 22 has a square protrusion 705 and FIG. 23 has a triangle-shaped protrusion 715.

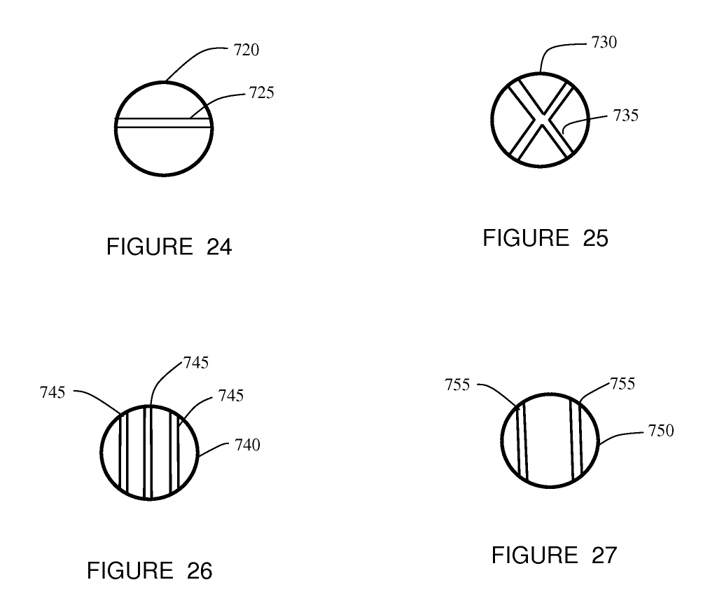

FIG. 24 is a top view of a thumb plate 720 that has a ridge 725 on the top surface. FIG. 25 is a top view of a thumb plate 730 having a raised “X” shape 735. FIG. 26 is a top view of a thumb plate 740 having an arrangement of three ridges 745. FIG. 27 is a top view of a thumb plate 750 having an arrangement of two ridges 755.

The raised elements on the thumb plates in FIGS. 16-27 may be formed and configured in various alternative ways, some already described above. Additional alternative configurations include variations in height of the feature with regard to the top surface of the thumb plate.

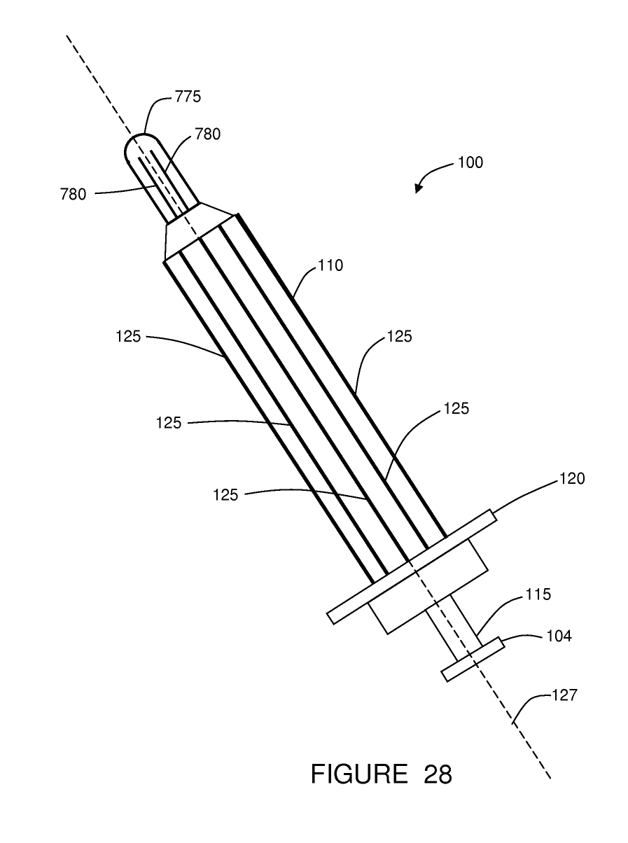

FIG. 28 is a side view of a syringe according to an alternative embodiment. The syringe 100 has a cylindrical tube 110. The tube 110 is also referred to as a “barrel.” The tube 110 includes a flange 120 at one end and an adapter at the other end. The adapter is not visible in this view. The adapter is covered by an adapter cap 775. The adapter cap 775 is also referred to as a hub cover or a plug cap. The adapter cap 775 covers the open end of the barrel 110. The adapter cap 775 is another location on the syringe that includes non-textual tactile elements to alert the user to the syringe contents. In this embodiment, the adapter cap 775 has a plurality of vertical ridges 780 or “ribs” similar to the plurality of vertical ribs 125 on the syringe barrel 110.

As described above with regard to the syringe barrel, the ribs 780 provide a tactile alert to the person using the syringe to deliver medication. The ribs 780 are a surface element on the adapter cover that differentiates the adapter cover of the present embodiment from a conventional adapter cover without tactile differentiation. The user of the syringe of the present embodiment would have a tactile alert when handling the ridged adapter cover beyond any text labeling. The tactile differentiation described above with regard to the syringe barrel may be applied similarly to the adapter cap. In one embodiment, the tactile differentiation elements on the adapter cap match that provided on the syringe barrel. In an alternative embodiment, the tactile differentiation on the adapter cap and on the barrel are different. In a further alternative embodiment, there is tactile differentiation on the adapter cap only and not on the syringe barrel. In a further alternative embodiment, the tactile differentiation elements on the syringe barrel, thumb plate and adapter cap match. This embodiment has the benefit of maximizing the alert to the user.

Other variations from the syringes described above include using different colors for the plunger seal to provide visual differentiation of the syringes.

Embodiments of the invention described above provide tactile labels that alert the users of syringes in a medical setting to particular contents of the syringes. This differentiation of the syringes with tactile labeling from conventional syringes and also from other syringes with tactile labeling provides a significant safety improvement in that easily distinguishable differences between syringes will discourage medicine delivery errors. Specifically, medications designated as “high-alert” medications such as muscle relaxants, narcotics, and insulin could be more easily distinguished from other medications. A differently configured syringe would be a tactile indicator to a health care provider, often working in an environment that is hurried and full of distractions, of the type of drug that the syringe holds. Further, syringe differentiation would be a strong safety enhancement in the production and use of pre-filled syringes which are expected to be used for a wide range of medications, including vaccines, anticoagulants, anti-infectives, anti-inflammatory agents, hematological agents, multiple sclerosis therapies, hormone therapies, obstetric agents, cancer therapies, and pain relievers.

It is to be understood that the above-identified embodiments are simply illustrative of the principles of the invention. Various and other modifications and changes may be made by those skilled in the art which will embody the principles of the invention and fall within the spirit and scope thereof.

We claim:

1. A barrel for a syringe, comprising:
a tube;
an outer surface of the tube; and
a tactile element located on the outer surface and configured such that the tactile element forms a non-textual label, wherein the tactile element comprises a plurality of vertical ribs arranged and configured to create an impression of a particular geometric figure in cross-section, whereby a user of the syringe is able to perceive an alert to contents of the syringe.

2. (canceled)

3. (canceled)

4. The barrel of claim 1 wherein the geometric figure is a square.

5. The barrel of claim 1 wherein the geometric figure is a triangle.

6. The barrel of claim 1 wherein the tactile element further comprises a plurality of circumferential ribs.

7. The barrel of claim 6 wherein the plurality of circumferential ribs are located and arranged as a plurality of groups on the outer surface.

8. The barrel of claim 1 wherein the tactile element further comprises a spiral ridge.

9. The barrel of claim 1 wherein the tactile element further comprises a plurality of bumps.

10. The barrel of claim 1 wherein the tactile element further comprises a plurality of dimples.

11. A syringe for delivery of a medication, comprising:
a barrel having a cylindrical tube, the cylindrical tube having an outer surface; and
a tactile element located on the outer surface and configured such that the tactile element forms a non-textual tactile label, the tactile element being a plurality of vertical ribs arranged and configured to create an impression of a particular geometric figure in cross-section, whereby a user of the syringe is able to perceive an alert to contents of the syringe.

12. (canceled)

13. The syringe of claim 11 wherein the geometric figure is a square.

14. The syringe of claim 11 wherein the geometric figure is a triangle.

15. A syringe for delivery of a medication, comprising:
a barrel having a cylindrical tube, the cylindrical tube having an outer surface:
a tactile element located on the outer surface and configured such that the tactile element forms a non-textual tactile label whereby a user of the syringe is able to perceive an alert to contents of the syringe; and
a plunger having a thumb plate, the thumb plate further including a second tactile element configured such that the second tactile element forms a second non-textual tactile label whereby a user of the syringe is able to perceive an alert to contents of the syringe.

16. The syringe of claim 15 wherein the second tactile element comprises a geometric configuration of the thumb plate.

17. The syringe of claim 15 wherein the thumb plate has an upper surface and the second tactile element comprises at least one raised structure located on the upper surface of the thumb plate.

18. The syringe of claim 11 further comprising an adapter cap, the adapter cap further including a third tactile element configured such that the third tactile element forms a third non-textual tactile label whereby a user of the syringe is able to perceive an alert to contents of the syringe.

19. A syringe for delivery of a medication, comprising:
a barrel having a cylindrical tube, the cylindrical tube having an outer surface; and
a tactile element located on the outer surface and configured such that the tactile element forms a non-textual tactile label whereby a user of the syringe is able to perceive an alert to contents of the syringe
a plunger having a thumb plate, the thumb plate further including a second tactile element configured such that the second tactile element forms a second non-textual tactile label; and
an adapter cap, the adapter cap further including a third tactile element configured such that the third tactile element forms a third non-textual tactile label,
wherein the tactile elements on the barrel, the thumb plate and the adapter cap match,
whereby a user of the syringe is able to perceive an alert to contents of the syringe from the non-textual tactile labels

20. The syringe of claim 11 wherein the tactile element is a flexible sheet with tactile labeling elements, the flexible sheet affixed to the outer surface of the tube.
